(19) United States
(12) Patent Application Publication
(10) Pub. No.: US 2026/0013756 A1
(43) Pub. Date: Jan. 15, 2026

(54) MICROFABRICATED IMPLANTABLE PROBES

(71) Applicant: ALCORIX CO., Plainfield, IL (US)

(72) Inventor: Nicolaie Moldovan, Plainfield, IL (US)

(21) Appl. No.: 19/249,355

(22) Filed: Jun. 25, 2025

Related U.S. Application Data
(60) Provisional application No. 63/670,631, filed on Jul. 12, 2024.

(51) Int. Cl. A61B 5/1486; A61B 5/00; A61B 5/145

(52) U.S. Cl. CPC A61B 5/14865; A61B 5/14546; A61B 5/6847; A61B 2560/0468; A61B 2562/166; A61B 2562/227

(57) ABSTRACT

The disclosure features a microfabricated implantable probe, the probe comprising (a) a semiconductor substrate microprobe body including a handling portion and an elongated shaft extending from the handling portion to a tip, the tip being configured for insertion into a target area of a subject mammal, (b) at least one pair of enzyme-functionalized sensing electrodes disposed on the shaft, (c) individually addressable conductive metallic leads extending for a specified distance from each sensing electrode to contact pads disposed on the handling portion, and (d) a polymeric separating layer configured to separate each sensing electrode in the pair of electrodes from the other sensing electrode. The disclosure also features methods of using the probe to detect neurochemicals.

### RELATED APPLICATION

This application claims priority and the benefits under 35 U.S.C. § 119 (e) to co-pending U.S. Provisional Application No. 63/670,631 filed on Jul. 12, 2024 and titled Improved Microfabricated Neurochemical Probe, which is herein incorporated by reference in its entirety.

### STATEMENT REGARDING FEDERALLY SPONSORED RESEARCH OR DEVELOPMENT

This invention was made with government support under Award Number 5R42NS115282-03 and FAIN: R41NS115282, both awarded by the National Institute of Health. The government has certain rights in the invention.

### BACKGROUND

Several brain disorders are linked to imbalances in gamma-amino-butyric acid (GABA) and Glutamate (GLU) homeostasis, with dysregulation of GLU and GABA occurring in neurological disorders such as epilepsy, dementia, Parkinson's Disease, schizophrenia, and addiction. A fundamental understanding of GLU and GABA dynamics in various brain regions would likely lead to a better understanding of human brain function and to the development of new and more effective treatments. Thus, work has been conducted to develop microprobe sensors for the detection of GLU and GABA levels in the brains of animal subjects.

U.S. Pat. No. 11,946,896 (Arumugam), incorporated herein by reference, describes a microprobe sensor for the simultaneous real-time amperometric detection of GABA and GLU in the presence of specific enzymes coating separate electrodes (Gabase and glutamate oxidase, respectively, embedded in a matrix material). The microprobe sensor is also sometimes referred to as “microbiosensor”. Measurements of the concentrations of these important neurochemicals can be conducted simultaneously through external contact pads on the microprobe body. An individual “sentinel” electrode without enzymatic activation, but bearing only the matrix material is included in the microprobe design in order to facilitate subtraction of background electrochemical detection of other electrochemically active analytes such as ascorbic acid. In Arumugam's approach, the individual GABA and GLU electrodes are relatively widely spaced (e.g., 1 mm) both due to the construction and to avoid their cross-contamination with the specific functionalization enzymes. This limits the minimum size of the microprobe, resulting in greater damage upon insertion into a subject and potentially affecting the sensitivity of detection.

Efforts to develop a microprobe sensor for the simultaneous real-time detection of GABA and GLU having a smaller size, to minimize damage upon insertion, are described in “Brain-implantable multifunctional probe for simultaneous detection of glutamate and GABA neurotransmitters”, Nicolaie Moldovan et al., Sensors and Actuators B: Chemical, Volume 337, 15 Jun. 2021, 129795, and “Brain-implantable multifunctional probe for simultaneous detection of glutamate and GABA neurotransmitters: Optimization and In Vivo Studies”, Nicolaie Moldovan et al., Micromachines 2022, 13, 1008.

### SUMMARY

The present disclosure features brain-implantable multifunctional probes, suitable for detection of neurotransmitters, that achieve a probe with small cross section, without sacrificing accuracy. Preferred probes are capable of reliable and sensitive simultaneous detection, with nano-molar sensitivity of multiple neurochemicals. In some implementations the probe has a very close spacing between adjacent neurochemical sensing electrodes, allowing GLU and GABA to be detected in substantially the same location in the subject. The close spacing of the electrodes also allows a very narrow probe, contributing, along with a probe thickness less than 0.25 mm, to a small insertion cross section, minimizing trauma to the subject.

In one aspect, the present disclosure features a microfabricated implantable probe, the probe comprising: (a) a semiconductor substrate microprobe body including a handling portion and an elongated shaft extending from the handling portion to a tip, the tip being configured for insertion into a target area of a subject mammal, (b) at least one pair of enzyme-functionalized sensing electrodes disposed on the shaft, (c) individually addressable conductive metallic leads extending from each sensing electrode to contact pads disposed on the handling portion, and (d) a polymeric separating layer configured to form an alveolae around each sensing electrode and prevent cross-contamination between the sensing electrodes during the functionalization process.

Some implementations include one or more of the following features.

The probe may also include an insulating layer, covering the leads and underlying the polymeric separating layer, to impede exposing the metal leads to the bodily environment. The sensing electrodes may be functionalized for neurochemical sensing. For example, the sensing electrodes may be configured to simultaneously and separately detect gamma-amino butyric acid (GABA) and glutamate (Glu). This may be accomplished by functionalizing one of the sensing electrodes with Gabase and the other with GOx enzyme for the sensing of GABA and GLU respectively.

In some implementations a spacing between the sensing electrodes is less than 100 microns, for example 70 microns or less, or even 20 microns or less if the leads are positioned beneath the electrodes by using multiple layers of metallization.

The polymeric separating layer may have a thickness of at least 5 microns, for example the thickness of the polymeric separating layer may be from about 5 to 20 microns, e.g., about 5 to 10 microns.

The polymeric separating layer may be formed of material selected from the group consisting of epoxies, polyimides, and parylenes. In some implementations the material may be an epoxy photoresist, for example SU-8 epoxy photoresist.

The probe may comprise two or more pairs of sensing electrodes, and adjacent pairs may be separated from each other along the length of the shaft by a sentinel electrode.

The probe may further comprise a plurality of pore openings disposed on the shaft, the pore openings being in fluid communication with microchannels configured to allow fluid (e.g., a calibration fluid or drug solutions) to flow from the on-chip micro-channels to tissue areas adjacent to the sentinel and/or sensing electrodes, e.g. for forming an on demand, on chip calibrator (odic) In some implementations the pore openings are surrounded by the polymeric separating layer, to prevent the penetration of the functionalization fluids into the microchannels during the functionalization process. Entry of the functionalization fluid into the pores/microchannels can cause clogging and thus is undesirable.

The polymeric separating layer may substantially cover an upper surface of the length of the shaft, i.e., cover the upper surface except for openings in the areas of the sensing electrodes, sentinel electrodes, and pore openings. This provides a generally smooth surface that minimizes dragging of tissue or bending of the probe by asymmetric drag. Covering the upper surface in this manner also provides a symmetrical distribution of polymer across the width of the shaft, to prevent twisting of the probe shaft by stress or insertion drag forces.

In some implementations the leads extend a predetermined distance of at least 3 millimeters (e.g., from 3 mm to 6 mm) from each sensing electrode to a corresponding one of the contact pads on the handling portion of the chip. This distance may be determined based on the thickness of the skull of a subject. At least 0.5 millimeter of the predetermined distance along the handling portion of the chip may be attached to a longer flexible electrical and microfluidic circuit.

In another aspect, the present disclosure features a method of detecting neurochemicals in an animal subject, the method comprising: (1) providing a probe, the probe comprising (a) a semiconductor substrate microprobe body including a handling portion and an elongated shaft extending from the handling portion to a tip, the tip being configured for insertion into a target area of the animal, (b) at least one pair of enzyme-functionalized sensing electrodes disposed on the shaft, (c) individually addressable conductive metallic leads extending from each sensing electrode to contact pads disposed on the handling portion, and (d) a polymeric separating layer configured to separate each sensing electrode in the pair of electrodes from the other sensing electrode; and (2) inserting the tip through a skull opening into the brain tissue of the subject.

The method may further include detecting the levels of GABA and GLU in the brain using data obtained from the sensing electrodes.

Besides Glu and GABA, other neurotransmitters can be detected with the same device, (e.g. Dopamine, Acetylcholine, etc.) by choosing different functionalization enzymes. Other neuroactive substances of particular interest would be those acting as agonist-antagonist pairs, which are of importance in neuromuscular diseases and drug addiction.

Within this specification embodiments have been described in a way which enables a clear and concise specification to be written, but it is intended and will be appreciated that embodiments may be variously combined or separated without parting from the invention. For example, it will be appreciated that all preferred features described herein are applicable to all aspects of the invention described herein.

### DESCRIPTION OF THE DRAWINGS

FIG. 1 is a schematic perspective view showing a microfabricated neurochemical probe according to one implementation.

FIG. 2 is a top planar view of the probe shown in FIG. 1.

FIG. 3 is an enlarged detail view of the tip portion of the probe shown in FIG. 1.

FIG. 4 is perspective view corresponding to the view shown in FIG. 3, further enlarged.

FIG. 5 is an even more highly enlarged detail view of the neurochemical sensing electrodes closest to the tip of the probe shown in FIG. 4.

FIG. 6 is similar to FIG. 4 but shows a probe that is an example of an alternative implementation having a different electrode configuration, for use in a different animal subject (mouse in this case).

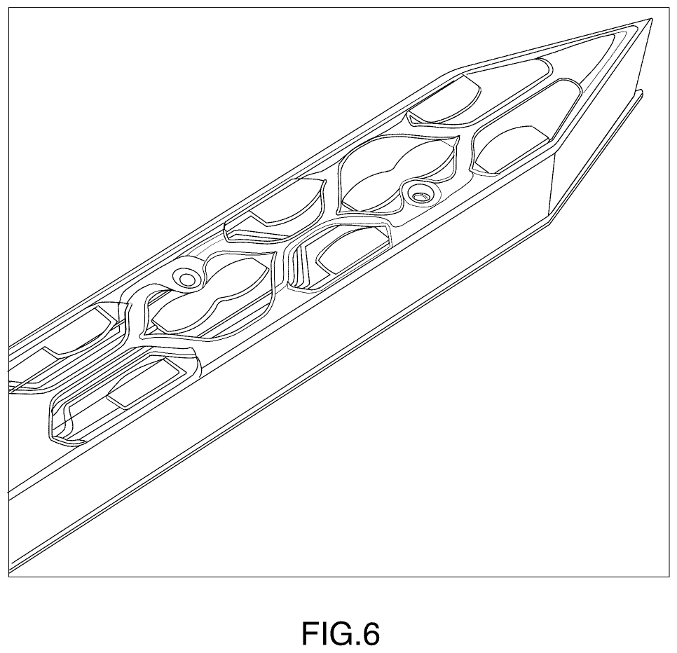

FIG. 7 is a diagrammatic perspective view showing a flexible circuit being assembled onto the probe shown in FIG. 2.

FIG. 8 is a diagrammatic view of a wafer including several different types of probes arranged to optimize the space available on the wafer.

FIG. 9 is a diagrammatic view of a pulled glass nanopipette applying a droplet of enzyme to an electrode to functionalize the electrode.

### DETAILED DESCRIPTION

In the probes disclosed herein, semiconductor fabrication technology has been applied to improve probe sensitivity and reliability for simultaneous detection of two different chemicals, e.g., the important neurochemicals Glutamate (GLU) and Gamma Amino Butyric Acid (GABA).

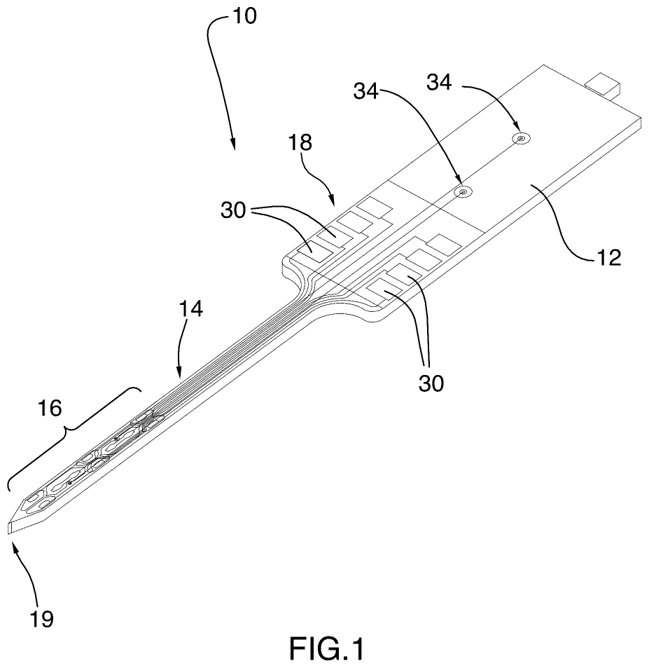

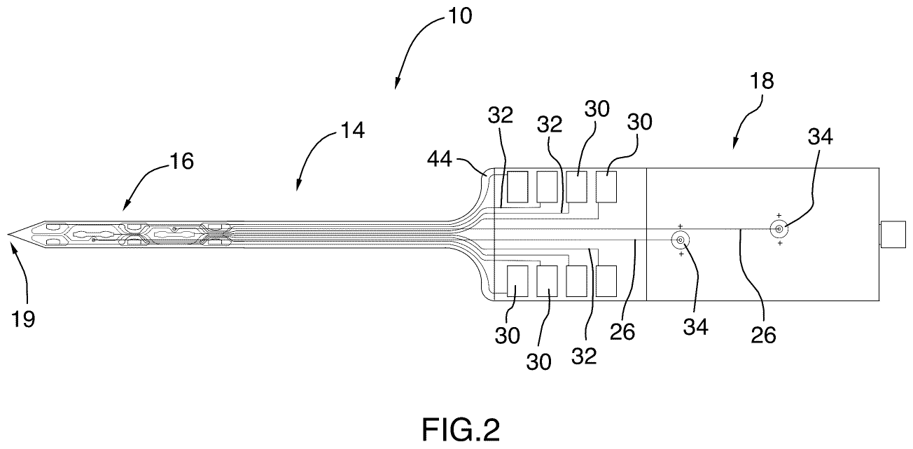

Referring to FIGS. 1-2, a probe 10 according to one implementation includes a silicon substrate 12, e.g., of single crystal silicon, that is shaped to form an elongated shaft 14 having a tip portion 16, configured for insertion into a subject, and a wider handling chip 18 configured to be manipulated by a user to insert the probe. The tip portion 16 terminates in a pointed tip 19. The silicon substrate 12 may have a thickness of, for example, 200 microns or less, for example from 100 to 200 microns, or 50 microns to 100 microns.

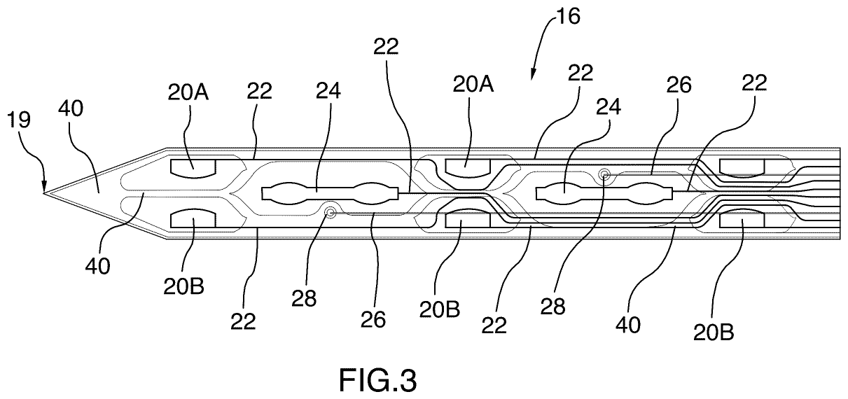

Referring to FIG. 3, the probe includes a plurality of pairs of neurochemical sensing electrodes 20A and 20B, and separate leads 22 that extend from each of the sensing electrodes 20A and 20B to provide electrical communication with contact pads 30 on the handling chip 18.

The pairs of sensing electrodes 20A,20B are positioned along the length of the tip portion 16, to allow separate simultaneous measurements of GLU and GABA at various depths of penetration of the probe shaft into the target neurological detection site. This can allow researchers to “map” in depth the area of the brain in which the probe is inserted.

Each pair of neurochemical sensing electrodes is functionalized with Gabase enzyme on one electrode and GOx enzyme on the other. As is known in the art, this functionalization causes the sensing electrodes to selectively produce hydrogen peroxide (H₂O₂) for the indirect electrochemical detection of the presence of GABA and GLU, respectively, via the generation of peroxide.

The enzymes may be provided, for example, in the form of gels or a binding matrix. The functionalization process is carried out before (e.g., hours, days, weeks or months) inserting the probes into the tissue. The sentinel electrodes, discussed in detail below, lack enzyme functionalization but are covered with the same matrix or gel as used in the functionalization.

The neurochemical sensing electrodes 20A,20B of each pair are very closely spaced. For example, in some implementations GABA and GLU electrodes in each pair are spaced less than about 100 microns apart at their closest approach, for example less than about 70 microns and in some cases about 50 to 70 microns apart. The spacing of the electrodes can be made even smaller by running the leads under the electrodes, e.g., by providing a layer of metallization that is etched to form the leads, covering this layer with silicon nitride, and then forming the electrodes and connecting the leads through the silicon nitride layer, a technique known as “multiple metallization layers” to those skilled in the art. With this arrangement the spacing of the electrodes can be 20 microns or less, or in some cases 15 microns or less. The spacing between electrodes in the case of multiple metallization layers is limited by the width of the polymeric separating layer between the electrodes. This close spacing allows GABA and GLU to be detected in substantially the same location in the subject, increasing the meaningfulness of the comparison of the GABA and GLU readings for a given pair of sensing electrodes.

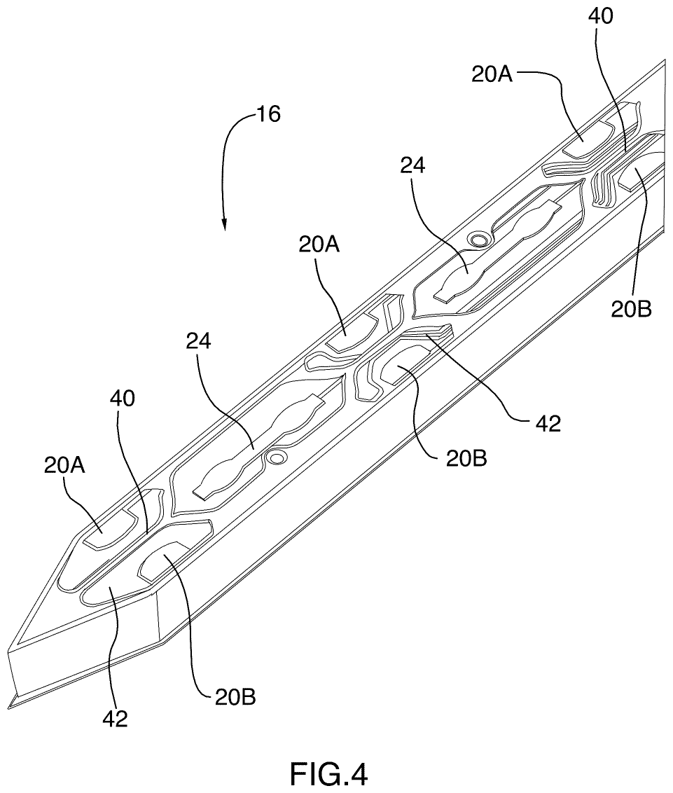

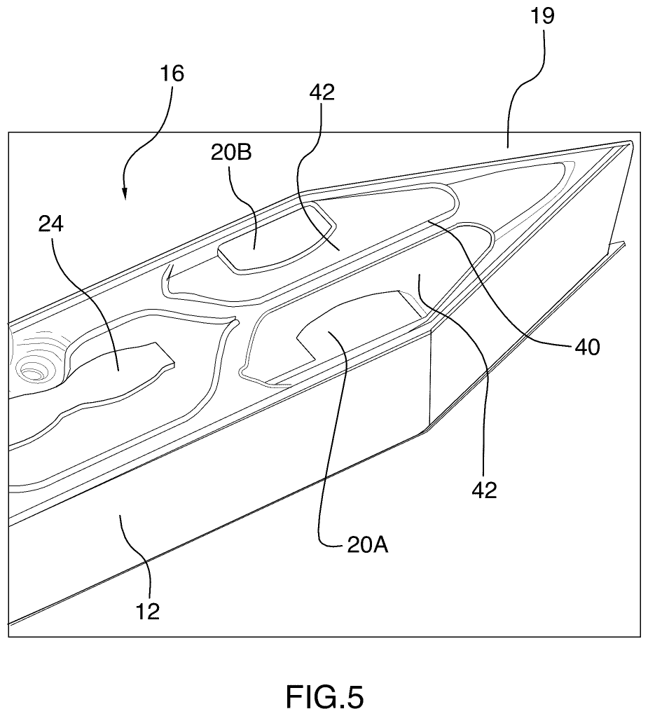

This close spacing can be achieved without cross-contamination between the sensing electrodes due to the presence of a polymeric separating layer 40 that covers the upper surface of the tip portion 16. The polymeric separating layer 40 creates a barrier between the closely spaced electrodes by defining recesses in which the sensing electrodes are disposed, as can be seen in FIGS. 4-5. As a result, the polymeric separating layer prevents migration of functionalization fluid between the sensing electrodes during the functionalization process, carried out ex-vivo. Because the separating layer is polymeric it can be patterned easily, e.g., using a single photolithography process, to form a relatively thick layer, and thus relatively deep recesses protecting the sensing electrodes from cross-contamination.

The polymeric separating layer is applied to the surface of the substrate 12 and then patterned directly by photolithography to expose the electrodes, forming recessed areas around the electrodes. The walls 40 (FIGS. 4 and 5) of these recessed areas, which are disposed on a top surface of SiNₓ areas 42 that surround the electrodes, serve as a fence, preventing capillary spreading of functionalization fluids from one electrode to an adjacent electrode. To form an effective fence against cross-contamination of the electrodes and pores during the functionalization process with enzyme-containing fluids, the polymeric separating layer generally should be at least 5 microns thick, for example about 5 to 20 microns thick. In some implementations the layer is from 5 to 10 microns thick. The thickness of the polymeric separating layer equates to the height of wall 40 where it forms an alveolae around the two electrodes 20A and 20B, allowing the functionalization fluid to be retained without spilling over from one electrode to the other during the functionalization process.

The polymeric separating layer 40 can be formed of many electrically and chemically inert materials. While a typical example is SU-8 epoxy-photosensitive polymer, other photosensitive polymers can be used, e.g., photosensitive polyimide films for direct patterning. Non-photosensitive polymers that are patternable by masked etching can also be used by forming a mask on top that is patternable by standard lithography processes.

One material of particular utility is SU-8 epoxy polymer, an epoxy-based negative photoresist. One of the characteristics of SU-8 that makes it suitable for use in this application is its high degree of biocompatibility. The SU-8 polymer separating layer is also easily fabricated due to its resistance to other chemicals used in the fabrication process. Spin-coating is performed on the wafer level, prior to separating the chips, when the pores 28 are sealed, in order to prevent penetration of polymer into the micro-channels 26. The pores 28 have to be opened within a deep-etch RIE process for punching through the thick microchannel-sealing SiN, using a thick positive photoresist (typically a novolac-based photoresist such as SPR220-7) patterned on top of the SU-8. SU-8 is resistant to solvents and developers of novolac resists, and to acetone (for removing the novolac photoresist). SU-8 also resists degradation during the Bosch process, used to shape the shafts and handling chips by deep-etching around them the Si. Moreover, because the SU-8 polymer is photosensitive it can be processed with standard lithography.

SU-8 photoresist includes Bisphenol A Novolac epoxy that is dissolved in an organic solvent, and a mixed Triarylsulfonium/hexafluoroantimonate salt as a photoacid generator. SU-8 photoresist is available commercially from, for example, Westlake Epoxy under the tradename EPON™ Resin SU-8.

In the implementation shown in FIGS. 1-3 the probe includes three pairs of GABA/GLU sensing electrodes 20A, 20B, with each electrode being less than about 150 microns in length (at its largest dimension), for example from about 50 to 150 microns. The spacing between the pairs of electrodes will depend upon the probe design and the intended test subject, corresponding to the spacing between cortical layers, but will often range from about 250 to 500 microns.

Referring to FIG. 3, the elongated shaft 14 of the probe also includes, in addition to the neurochemical sensing electrodes 20A,20B, sentinel electrodes 24 and microfluidic sealed microchannels 26 having corresponding outlet pore openings 28.

In the embodiment shown in FIGS. 1-3, adjacent pairs of sensing electrodes are separated by longer sentinel electrodes 24, thus allowing the same sentinel electrode to be shared for two consecutive pairs of sensing electrodes. Alternatively, triplets of probes (GABA probe, Glu probe and sentinel electrode) can be grouped closer, and measurement can be performed such that GABA and Glu are obtained locally from the GABA and Glu electrodes, with the local non-specific signal being detected by the proximal sentinel electrode. While this triplet-based design has the advantage of locally sensing all the components, a probe with multiple triplets needs a more crowded wiring, creating possibilities for higher capacitive or inductive cross talk, manifested in noisy signals.

The sentinel electrode, although not functionalized, can be coated with a matrix polymer, such as the polymer used in the sensing electrodes to fix in place the GABase or Glutamate oxidase, but without these chemicals. The non-functionalized sentinel electrodes should be in close proximity to the GABA and GLU electrodes they serve (or can be interposed at equal spacing between two pairs of GLU/GABA detection electrodes) to allow higher sensitivity through subtraction of amperometric signals from electrochemically active chemicals that are not activated by the enzymes on the electrodes, e.g., ascorbic acid. The sentinel electrodes are configured to allow a baseline level of hydrogen peroxide to be measured. The sentinel electrodes are typically at least 300 microns in length, and in some implementations have a length of, for example, 200 to 400 microns (largest dimension). The size and shape of the sentinel electrodes depends on the sensing organization on the probe. If the sensing sites are organized as electrode triplets, the sentinel electrodes can have any shape suited the probe layout (including round, square, elliptical etc.) but if the probes are organized to share sentinel electrodes between two pairs of GABA/Glu sensing electrodes, as shown, the sentinel electrode should generally have a slightly elongated shape to be in symmetrical proximity to the two pairs of GABA/Glu electrodes.

In general, the sentinel electrodes should be larger than the sensing electrodes. This is because the non-specific signals (i.e. H₂O₂ not derived from Glu or GABA decomposition) are weaker and require a higher sensitivity for detection, with the sensitivity being proportional to the detector electrode area. The correction of the Glu and GABA signals obtained with the smaller active electrodes can be calculated considering the electrode area ratio and the signals measured during a calibration process, when solutions of H₂O₂ are injected in the proximity of the probe pairs (or triplets) for calibration purpose. Limiting factors for the size of the sentinel electrodes are that the sentinel electrodes should be small enough to not investigate too large a region, and the overall size of the probe should be small enough to minimize tissue damage to the subject.

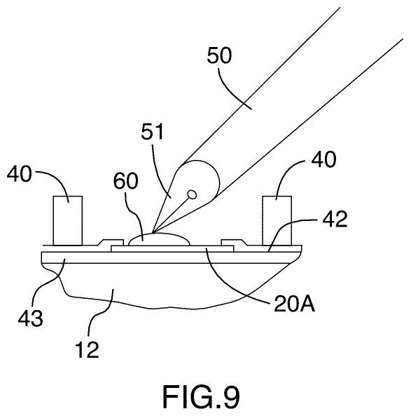

In the embodiment shown in FIGS. 1-3, in which there are shared sentinel electrodes, there are a total of six sensing electrodes and two sentinel electrodes interposed between the pairs of sensing electrodes. As shown in FIG. 3, each of these eight electrodes has a lead 22. The leads 22 are in electrical communication with corresponding leads 32 (FIG. 2) which are in turn in electrical communication with corresponding contact pads 30 on the chip portion 18 of the probe. The leads 22 to 32 are covered by an insulating layer 43 (FIG. 9) such as SiNₓ, to prevent electrical short circuits by bodily fluids and pick up of spurious signals. This structure allows all the electrodes (sensing and sentinel) to be individually contacted and addressable.

A plurality of inlet pore opening 34 allow the flow of calibrated solutions of hydrogen peroxide to be injected from external sources during the in situ calibration process. From each pore opening 34 calibrated solution flow into and through a microchannel 26. The calibrated solution exits the microchannel 26 through an outlet pore opening 28 disposed adjacent a corresponding sentinel electrode 24. The injection of calibrated hydrogen peroxide solutions into the neural tissue in the proximity of the sensing and sentinel electrodes is used prior to neurotransmitter measurements to calibrate the electrical output of the sentinel electrodes in reaction to small local concentration variations in hydrogen peroxide produced by the enzymatic decomposition of neurotransmitters. Because the microchannels allow fluidic access to the corresponding sentinel electrodes, they facilitate use as on demand, in situ calibration means (ODIC).

The inlet and outlet pore openings 34 and 28 have a diameter of about 8 to 12 microns. Each pore opening is spaced about 8 to 12 microns from the corresponding sentinel electrode and is in fluidic connection with the buried microchannels along the shaft and a portion of the handling chip 18. The cross sectional dimensions of the buried microchannels may be, for example, between 10 μm by 10 μm and 15 μm by 20 μm.

The polymeric separating layer 40 extends over substantially the entire upper surface of at least the tip portion 16 of the probe, and in some cases the entire length of the shaft 14, terminating at end 44 shown in FIG. 2. Accordingly, the polymeric separating layer 40 also provides isolation from contamination with functionalization substances for the sentinel electrodes 24 and the outlet pore openings 28, as can be seen in FIGS. 4 and 5.

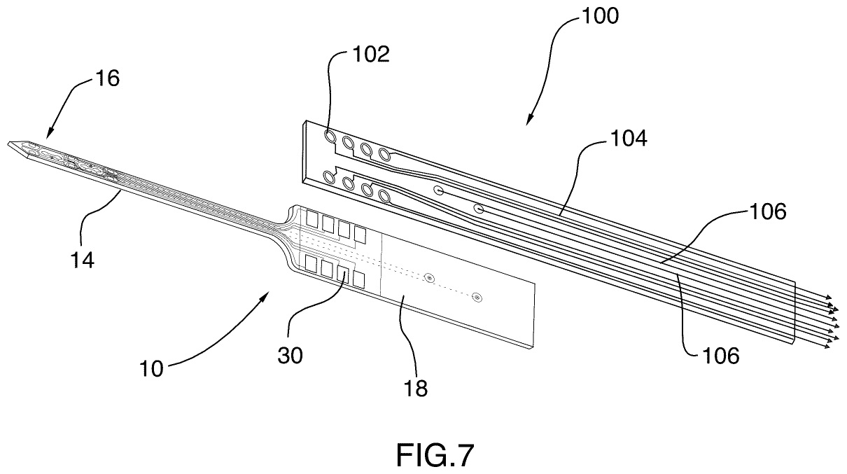

The dimensions of the probe 10 will vary depending on various factors, including the type of subject into which the probe will be inserted. However, as an example, the tip portion 16 of the shaft may have a length of from about 2 to 4 mm, a thickness of less than 0.25 mm, e.g., from 0.05 mm to 0.20 mm, and a width of less than 0.5 mm, e.g., from 0.1 to 0.3 mm, with the sharpened tip 19 having a length of from 0.25 to 0.35 mm. The handling chip 18 is wider than the shaft and is configured to be grasped by a user or with specialized equipment, and to be wide enough to accommodate an array of contact pads, allowing the attachment of a flexible electrical and microfluidic circuit 100 (FIG. 7). In some implementations the handling chip 18 is from about 2 to 5 mm long and from about 0.8 to 1.5 mm wide. The handling chip can be larger if desired, for example to assist in training users who are inexperienced with manipulating the small probes, since the handling chip will not be inserted into the subject.

The probe is formed using well known semiconductor fabrication techniques. The microchannels are obtained by trench etching, undercutting a masking layer and sealing with Silicon Nitride (SiNₓ) through joining the edges of the masking layer along the trenches, and are integrated into the silicon structure parallel to the electrical leads 22. Covering the lumens of the microchannels with the sealing layer of SiNₓ helps to lower the contact angle with the water-based solutions passing through the microchannels and thus allows a better capillary penetration of fluid. While in some implementations the masking layer and sealing layers are SiNₓ obtained through LPCVD processes, other material layers can also be used, such as SiO₂, SiC, amorphous carbon, or diamond, mixtures of them or composites, or any other electrically insulating layers deposited through isotropic processes. The neurochemical sensing electrodes 20A and 20B are then deposited, for example using electron beam evaporation, on an adhesion layer (e.g., chromium or titanium) on the silicon substrate 12. Leads 22 are also deposited during the electrode deposition step. The sensing electrodes may be formed of electrochemically roughened platinum, for example black platinum for ease of subsequent functionalization.

Obtaining the black platinum is an electrochemical process requiring all electrodes to be connected and form a large cathode. Transforming regular platinum into black platinum generally should be done considering ways to interconnect all probes and electrodes on a wafer scale. Alternatively, the electrodes of singular neuroprobes can be blackened after their release from the wafer and mounting of wiring attachments (e.g. FIG. 7) and short cutting all wire connections temporarily, to form a singular cathode.

The metallization of the electrodes and leads may be sealed with a thin (e.g., 1 micron) layer of e.g., chemical vapor deposited SiNₓ, and then etched to re-expose the electrodes and leads. Leads 22 and 32 are buried under an insulating layer (not shown, for clarity). The insulating layer may be, for example, Silicon nitride (SiN) or SiO₂, SiC, amorphous carbon, diamond, mixtures thereof, composites, or polyimide. The insulating layer provides a protective layer for both the electrical lead connections between the sensing/sentinel electrodes and their corresponding individually addressable contact pads on the body of the microprobe.

These semiconductor processing steps are described in further detail in the Microfabrication section of “Brain-implantable multifunctional probe for simultaneous detection of glutamate and GABA neurotransmitters”, Nicolaie Moldovan et al., Sensors and Actuators B: Chemical, Volume 337, 15 Jun. 2021, 129795, p. 2, the full disclosure of which is incorporated herein by reference.

The polymeric separating layer is then formed by spin coating the wafer and baking, prior to etching pore openings towards the micro-channels, to avoid penetration of polymer into the micro-channels.

If the polymer is photosensitive (e.g. SU-8 photoresist), the polymer can then be photolithographically patterned to form separation walls between alveoli on top of the electrodes.

Functionalization of the sensing electrodes is performed by applying, using capillary action or microspotting, a very small amount of enzyme in a matrix into each of the alveoli hosting a sensing electrode (i.e. Gabase+matrix onto the GABA electrode, Glu-oxidase+matrix onto the Glu electrode, and only matrix onto the sentinel electrodes). The functionalization substances are in fluid form during application, for example in a solution of bovine serum albumin and glutaraldehyde in deionized water, then transform into a gel matrix with embedded enzymes upon drying. For exemplification and not for limitation, application by capillary action can be performed, for example, using a nanopipette that is formed by locally heating and pulling a glass capillary tube until breaking, to form a narrow-ended tube with an outer diameter less than the polymer-fenced alveolae width, typically from 2 microns to 5 microns. The end of the pulled nanopipette can be machined using focused ion beam (FIB) milling into a pen-like tip, to allow the fluid to leak from the tube along a trench, to the contact point with the electrode to be coated. FIG. 9 shows an example of a pulled glass nanopipette 50 having a FIB-machined pen-like end 51. In FIG. 9 a functionalization fluid droplet 60 is being deposited with the nanopipette.

Referring to FIG. 7, a flexible circuit 100, fabricated from sandwiched and thermally bonded polyimide layers, is assembled with probe 10. Embedded metal contact electrodes 102 of the flexible circuit 100 are bonded to the contact pads 30 on the chip pad 18 of probe 10. The flexible circuit contains embedded electrical leads 104 and fluidic microchannels 106 which provide electrical and microfluidic connections, respectively. The use of a flexible circuit, rather than a printed circuit board, makes the probe assembly more flexible, accommodating tissue micro-motion during live animal tests. The flexible circuit also reduces the physiological impact during chronic use, by adapting the probe flexibility and compliance to brain tissue micromotion. The flexible circuit may have a thickness of at least 10 microns, for example from about 10 to 20 microns. Polymer O-rings may be provided around the inlet pores 34 of the handling chip 18 to allow snap-in alignment of the flexible circuit 100. After bonding, the handling portion of the chip and the portion of the flexible circuit atop can be covered or embedded in an additional resin, e.g., polyimide, epoxy resins or other, for better insulation, sturdiness of the bond, and easier handling.

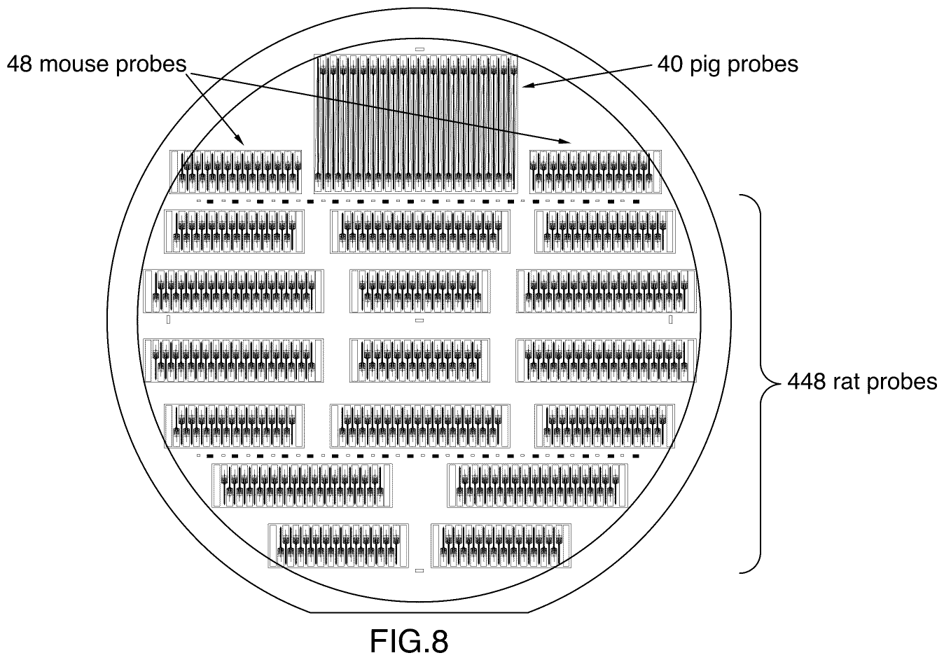

Referring to FIG. 8, if desired a number of arrays of different types of probes (for example, probes of different sizes for mice, rats, and pigs) can be manufactured on a single wafer. This will generally reduce the area of the silicon wafer that is not utilized, saving cost and reducing waste.

### OTHER EMBODIMENTS

A number of embodiments have been described. Nevertheless, it will be understood that various modifications may be made without departing from the spirit and scope of the disclosure.

For example, the probe can be configured differently to adapt it for use with different subjects. The arrangement of the pairs of sensing electrodes and sentinel electrodes along the probe shaft, their size, geometry, and/or number, can be adjusted for different subjects, for example, rats, mice, birds, pigs, sheep, monkeys, primates and other animals used in testing. The sensing and sentinel electrodes can also be generalized for other neurochemicals or other analytes.

An example of a generalization of the probe designs described herein would include further pairs of GABA/GLU sensing electrodes with interposing sentinel electrodes and corresponding microchannels with pore openings. For example, four pairs of GABA/GLU sensing electrodes would be interspersed with three sentinel electrodes with each sentinel electrode between two pairs of GABA/GLU sensing electrodes or five pairs of GABA/GLU sensing electrodes with interposed sentinel electrodes, etc., Each additional pair of GABA/GLU sensing electrodes would be separated from the other sensing electrodes by another sentinel electrode.

Also, additional electrodes can be provided on the opposite side of the shaft, either in the same arrangement or in a different arrangement. Such an arrangement would allow discerning between GABA or Glu emissions from neurons on one side or the other of the probe, contributing to the better neuronal mapping of the neurotransmitter generation. The way the signals on the two faces fade away can also be significant for neuroscientists. Moreover, probe pairs (or triplets) on the two sides can be intercalated, allowing a finer vertical mapping. More probes on the same shaft can also be useful in detecting other neurochemicals, simultaneous with GABA and Glu, which could benefit other types of studies (e.g. addiction).

Another alternative embodiment (not shown) would include two, three, or more microchannels and pore openings of similar sizes and spacings in communication with each sentinel electrode and/or pair of sensing electrodes, or triplets of probe electrodes.

In some cases, the shaft can taper gradually from its narrow width along the length of the tip portion to the width of the handling chip, rather than the shaft being the same width over its entire length. This can help prevent breakage of the shaft.

While the probes are generally intended for use in non-human mammals, the probes could be used in humans (for example during acute experimentation in the course of brain surgery) or in other non-mammalian animal subjects (e.g. birds).

The probes could also be used for measuring chemicals other than GABA/GLU, in which case the electrodes would be functionalized to detect other substances. The specific geometry of the described probes (i.e. pairs of sensing probes and sentinel probes, accompanied by micro-channels with pore openings on the shaft to introduce substances in neural tissue) can find application in different parts of the brain (cerebellum, pituitary gland, spinal cord, larger nerves, or even hormonal glands of lymph nodes), the only restrictions being accessibility of the tissue and ease of penetration.

Accordingly, other embodiments are within the scope of the following claims.

What is claimed is:

1. A microfabricated implantable probe, the probe comprising:
(a) a semiconductor substrate microprobe body including a handling portion and an elongated shaft extending from the handling portion to a tip, the tip being configured for insertion into a target area of a subject mammal,
(b) at least one pair of enzyme-functionalized sensing electrodes disposed on the shaft,
(c) individually addressable conductive metallic leads extending from each sensing electrode to contact pads disposed on the handling portion, and
(d) a polymeric separating layer configured to form an alveolae around each sensing electrode and prevent cross-contamination between the sensing electrodes during the functionalization process.

2. The probe of claim 1 wherein the sensing electrodes are functionalized for neurochemical sensing.

3. The probe of claim 2 wherein the sensing electrodes are configured to simultaneously and separately detect gamma-amino butyric acid (GABA) and glutamate (Glu).

4. The probe of claim 3 wherein one of the sensing electrodes is functionalized with Gabase and the other sensing electrode is functionalized with GOx enzyme for the sensing of GABA and GLU respectively.

5. The probe of claim 1 wherein a spacing between the sensing electrodes is less than 100 microns.

6. The probe of claim 5 wherein the spacing is less than 70 microns.

7. The probe of claim 5 wherein the polymeric separating layer is formed of a material selected from the group consisting of epoxies, polyimides, and parylenes.

8. The probe of claim 1 wherein the polymeric separating layer has a thickness of at least 5 microns.

9. The probe of claim 8 wherein the thickness of the polymeric separating layer is from about 5 to 20 microns.

10. The probe of claim 1 wherein the polymeric separating layer is formed of a photosensitive material.

11. The probe of claim 1 wherein the polymeric separating layer comprises an epoxy photoresist.

12. The probe of claim 1 wherein the epoxy photoresist comprises SU-8 epoxy.

13. The probe of claim 1 wherein there are at least two pairs of sensing electrodes and adjacent pairs are separated from each other by a sentinel electrode.

14. The probe of claim 13 wherein the sentinel electrode is surrounded by a wall defined by the polymeric separating layer, isolating the sentinel electrode from the sensing electrodes.

15. The probe of claim 1 further comprising a plurality of pore openings disposed on the shaft, the pore openings being in fluid communication with microchannels configured to allow fluid to flow from the pore openings to the sensing electrodes.

16. The probe of claim 15 wherein the pore openings are surrounded by the polymeric separating layer.

17. The probe of claim 1 wherein the polymeric separating layer substantially covers an upper surface of the length of the shaft, except openings above the electrodes.

18. The probe of claim 1, wherein the leads extend a predetermined distance of at least 3 millimeters from each sensing electrode to a corresponding contact pad.

19. The probe of claim 18, wherein at least 1 millimeter of the predetermined distance is attached to a flexible electrical and microfluidic circuit.

20. The probe of claim 1, where the shaft has a thickness less than 0.25 mm.

21. A method of detecting neurochemicals in an animal subject, the method comprising:
providing a probe, the probe comprising
(a) a semiconductor substrate microprobe body including a handling portion and an elongated shaft extending from the handling portion to a tip, the tip being configured for insertion into a target area of the animal,
(b) at least one pair of enzyme-functionalized sensing electrodes disposed on the shaft,
(c) individually addressable conductive metallic leads extending for a specified distance from each sensing electrode to contact pads disposed on the handling portion, and
(d) a polymeric separating layer configured to separate each sensing electrode in the pair of electrodes from the other sensing electrode; and
inserting the tip through a skull opening into the brain of the subject.

22. The method of claim 21 further comprising detecting the levels of GABA and GLU in the brain using data obtained from the sensing electrodes
